(19) United States
(12) Patent Application Publication
(10) Pub. No.: US 2026/0335603 A1
(43) Pub. Date: Oct. 29, 2026

(54) Micro Puncture System for Transjugular Intrahepatic Portosystemic Shunt

(71) Applicant: University of Washington, Seattle, WA (US)

(72) Inventor: Feng Zhang, Seattle, WA (US)

(21) Appl. No.: 19/106,311

(22) PCT Filed: Aug. 29, 2023
(86) PCT No.: PCT/US2023/031327

§ 371 Date: Feb. 25, 2025

Related U.S. Application Data
(60) Provisional application No. 63/401,985, filed on Aug. 29, 2022.

(51) Int. Cl. A61M 25/06; A61M 25/01; A61M 25/09

(52) U.S. Cl. CPC A61M 25/0606; A61M 25/0136; A61M 25/09; A61M 2025/09008; A61M 2205/584

(57) ABSTRACT

The present disclosure provides a device comprising a catheter having a first end and a second end opposite the first end. The catheter includes a first lumen, a second lumen, and a third lumen. The first lumen is configured to receive a guidewire therethrough to position the device in a desired anatomy. The device includes a needle movably positioned within the second lumen of the catheter. The needle includes a first end and a second end opposite the first end, and the needle is hollow such that the needle includes a lumen extending from the first end to the second end. The needle is configured to transition from a first position in which the first end of the needle is positioned entirely within the second lumen of the catheter to a second position in which the first end of the needle extends out of the second lumen of the catheter.

### CROSS-REFERENCE TO RELATED APPLICATIONS

This application claims priority to U.S. Provisional Patent Application No. 63/401,985, filed Aug. 29, 2022, the contents of which are hereby incorporated by reference in their entirety.

### BACKGROUND

Unless otherwise indicated herein, the materials described in this section are not prior art to the claims in this application and are not admitted to be prior art by inclusion in this section.

Globally, cirrhosis caused more than 1.32 million deaths in 2017 and cases of decompensated cirrhosis increased to over 10.6 million. The mortality and morbidity resulting from decompensated cirrhosis increase sharply and the 1-year case-fatality rate can be as high as 80%. In 2017, 513,134 patients have decompensated cirrhosis, which caused 62,943 deaths in the USA. Esophageal varices are present in up to 85% of patients with decompensated cirrhosis and rupture at a rate of 10%-15% per year, resulting in life-threatening haemorrhage and a 6-week mortality around 20%.

A transjugular intrahepatic portosystemic shunt (TIPS) is an intraparenchymal artificial shunt created between the hepatic vein (HV) and the portal vein (PV) to alleviate portal hypertension, which is the most effective life-saving procedure to stop acute variceal bleeding (AVB) that cannot be controlled endoscopically and bring about threefold reduction in risk of bleeding recurrence. Pre-emptive (also called early) TIPS (p-TIPS) has been recommended as a rescue treatment in patients with AVB at high risk of treatment failure. p-TIPS achieved a better control of variceal bleeding, lower risk of rebleeding, better control of ascites, and an improvement in survival. Other acknowledged indications include refractory ascites, Budd-Chiari syndrome (BCS), hepatorenal syndrome, hepatic hydrothorax, portal hypertensive gastropathy, etc. Randomized controlled trials demonstrated a significant improvement of survival and 7.1-fold decrease in the risk of recurrence of tense ascites after TIPS. TIPS creation improves renal dysfunction in chronic liver disease. Patients with poorer renal function benefit the most from TIPS creation. The greatest evolution of TIPS in the past decades is the implementation of polytetrafluorethylene (PTFE)-covered stents, which significantly decreased shunt failure and improved clinical outcomes.

However, TIPS creation remains one of the most challenging and radiation intensive procedures, carrying high risk of various complications in up to 20% of cases even in the hands of experienced Interventional Radiologists. The most anxiety-provoking, high-risk and rate-limiting step in TIPS is the ‘blind puncture’ of the portal vein. Needle pass-related capsular perforation occurs in up to 33% of cases, with life-threatening peritoneal haemorrhage in 1%-2% of cases. Intraperitoneal haemorrhage caused by extrahepatic portal vein rupture is the most dangerous major complication of TIPS creation. Unfortunately, anatomical studies demonstrate that around 50% of portal vein bifurcations are extrahepatic. As the number of needle passes for portal venous access increases, the incidence of nontarget organ injury also increases. Moreover, a suboptimal position of the TIPS stent is associated with an increased risk for shunt dysfunction. Studies have shown that only 6.8% of patients who were eligible for early TIPS underwent the procedure because of the lack of availability of TIPS, especially outside academic expert centers. Although most cases of cirrhosis are in developing countries, the unavailability of TIPS is much more severe than developed nations due to the scarcity of expert interventional radiologists for this challenging life-saving technique.

The standard approach and complication rate have not been changed since TIPS was invented by pioneers in the field of interventional radiology (IR). Many researchers have described various methods aiming to reduce procedural time, radiation exposure, and risk of complications, but the fundamentals of the techniques and devices used for TIPS creation remain the same. The use of intravascular ultrasound (IVUS) via the inferior vena cava (IVC) facilitates access into the portal vein. However, no studies have shown that the current IVUS guidance reduces complication rates, fluoroscopy time or number of needle passes.

In children, TIPS has been employed to bypass sinusoidal and postsinusoidal obstruction of portal venous flow to alleviate symptoms of refractory ascites and variceal bleeding. Etiologies include cavernous transformation of the portal vein, congenital hepatic fibrosis, biliary atresia, and cystic fibrosis-related liver disease etc. TIPS is more difficult in children which needs longer procedure time, typically ranging from 2 to 6 hours and resulting from an increased difficulty puncturing small portal veins. In particular, biliary atresia is the most common cause of cirrhosis in children and the most common indication for pediatric TIPS. Despite very small portal veins associated with this condition, only adult sets are available for TIPS in pediatric patients. Accordingly, an improved system and method for TIPS procedures may be desirable.

### SUMMARY

The present disclosure provides devices and methods related to a needle-integrated balloon catheter system, which can be used for the safe, simple, effective, and precise creation of an intraparenchymal artificial shunt between the hepatic vein (HV) and the portal vein (PV) to alleviate portal hypertension, specifically named as Transjugular Intrahepatic Portosystemic Shunt (TIPS). Embodiments of devices and methods described herein may be adapted for locoregional delivery of therapeutics (radiofrequency-induced thermal energy, laser, chemotherapeutic drugs, cancer therapeutic gene, virus etc.) to intraluminal cancers, such as esophageal cancer, colorectal cancer, bile duct cancer, central lung cancer etc. and extraluminal malignancies, such as lymph node metastasis, prostate cancers, and pancreatic cancer, etc. Further, this system can be used for biopsy of hard-to-access intraluminal or extraluminal lesions, through large vessels such as IVC, and hollow organs, such as bile duct, esophagus, and trachea-bronchus, as non-limiting examples.

In particular, in a first aspect, the present disclosure provides a device comprising a catheter having a first end and a second end opposite the first end. The catheter includes a first lumen, a second lumen, and a third lumen. The first lumen is configured to receive a guidewire therethrough to position the device in a desired anatomy. The device also includes a needle movably positioned within the second lumen of the catheter. The needle includes a first end and a second end opposite the first end, and the needle is hollow such that the needle includes a lumen extending from the first end to the second end. The needle is configured to transition from a first position in which the first end of the needle is positioned entirely within the second lumen of the catheter to a second position in which the first end of the needle extends out of the second lumen of the catheter. The needle has a non-zero radius of curvature in the second position. The device also includes a balloon fixed to the first end of the catheter. The balloon is in fluid communication with the third lumen. The device also includes a handle coupled to the second end of the catheter.

In a second aspect, the present disclosure provides a device comprising a catheter having a first end and a second end opposite the first end. The device also includes a needle having a first end and a second end opposite the first end. The needle is configured to be removably positioned within the lumen of the catheter. The needle is configured to transition from a first position in which the first end of the needle extends out of the lumen of the catheter to a second position in which the first end of the needle is positioned in the lumen of the catheter. The device also includes a rigid hollow member fixed to and surrounding the needle adjacent to the first end.

In a third aspect, the present disclosure provides a system include the device of the first aspect and the device of the second aspect.

In a fourth aspect, the present disclosure provides a method for gaining access to a portal vein from a hepatic vein, the method comprising: (a) positioning a first guidewire in the hepatic vein of a patient, (b) moving the device of the first aspect over the first guidewire to the hepatic vein, (c) inflating the balloon to fix the device with respect to the hepatic vein, (d) transitioning the needle from the first position to the second position such that the first end of the needle moves through the liver of the patient and enters the portal vein, and (e) positioning a second guidewire through the lumen of the needle and into the portal vein.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a simplified block diagram containing an overview of the components of a device, according to an example embodiment.

FIG. 2A illustrates a side view of the device of FIG. 1, according to an example embodiment.

FIG. 2B illustrates a side view of the distal end of the device of FIG. 1 with the needle in the first position, according to an example embodiment.

FIG. 2C illustrates a side view of the distal end of the device of FIG. 1 with the needle in the second position and the balloon inflated, according to an example embodiment.

FIG. 3 illustrates a perspective view of the device of FIG. 1 including a fiber optical borescope, according to an example embodiment.

FIG. 4 illustrates a simplified block diagram containing an overview of the components of a device, according to an example embodiment.

FIG. 5A illustrates a side view of the distal end of the device of FIG. 4, according to an example embodiment.

FIG. 5B illustrates a side view of the distal end of the device of FIG. 4 with the catheter removed, according to an example embodiment.

FIG. 5C illustrates a close-up side view of the distal end of the device of FIG. 4 with the catheter removed, according to an example embodiment.

FIG. 6 illustrates a flowchart of a method, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

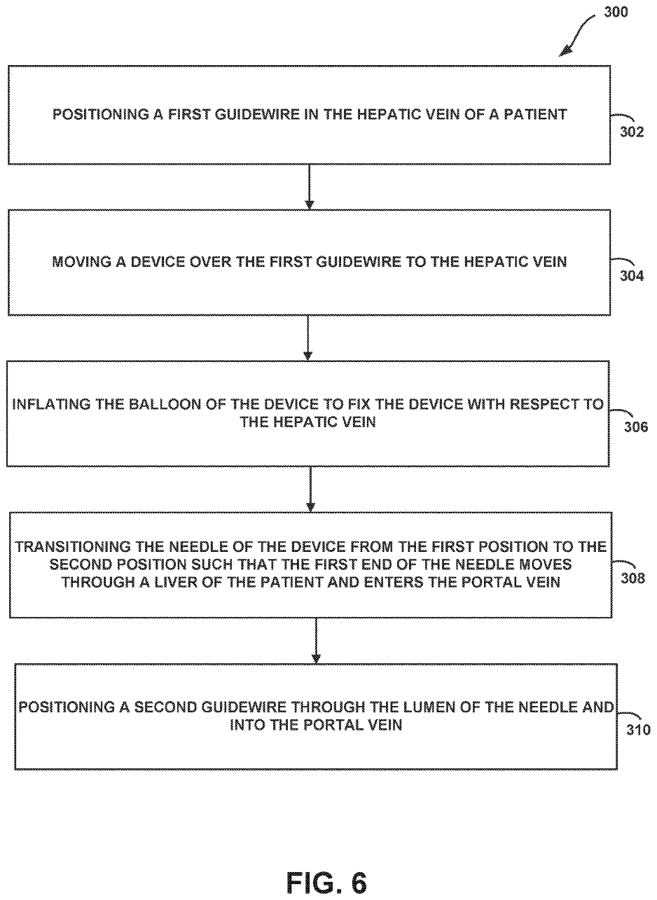

In FIG. 6, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 6 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Reference to, e.g., a “first” item does not require or preclude the existence of, e.g., a “second” or higher-numbered item. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means +/−5%.

As used herein, with respect to measurements, “substantially” means +/−5%.

Generally, the present disclosure provides a needle-integrated balloon catheter system, which can be used for the safe, simple, effective, and precise creation of an intraparenchymal artificial shunt between the hepatic vein and the portal vein to alleviate portal hypertension.

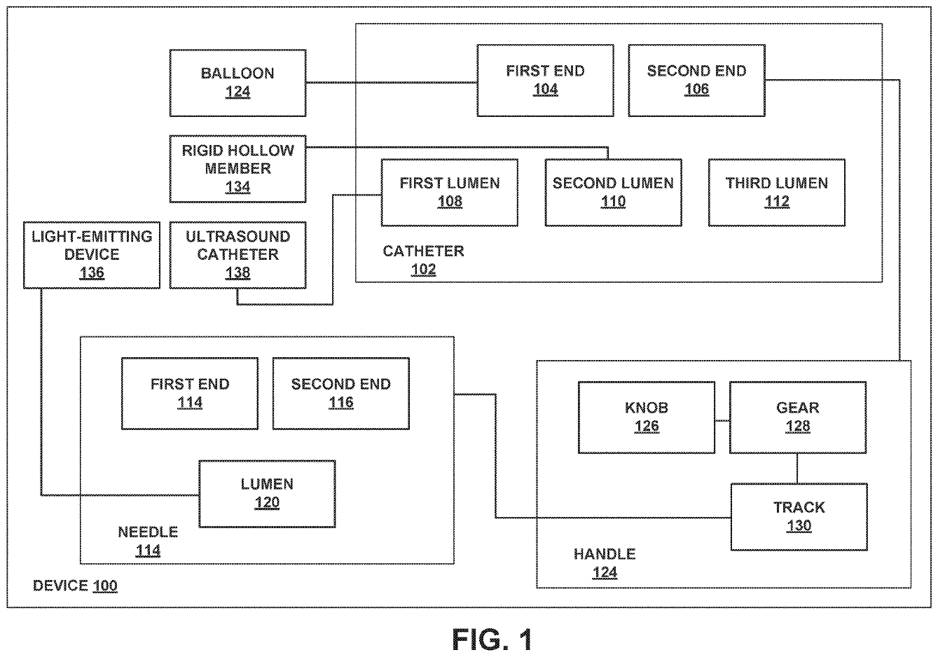

With reference to the Figures, the present disclosure provides a device 100 designed to gain access to a portal vein from a hepatic vein. In particular, FIG. 1 illustrates a simplified block diagram containing an overview of the components of a device 100, according to an example embodiment. As shown in FIG. 1, the device 100 includes a catheter 102 having a first end 104 and a second end 106 opposite the first end 104. The catheter 102 includes a first lumen 108, a second lumen 110, and a third lumen 112. In one example, each of the first lumen 108, the second lumen 110, and the third lumen 112 extend from the first end 104 to the second end 106 of the catheter 102. In use, the first lumen 108 is configured to receive a guidewire therethrough to position the device 100 in a desired anatomy. In one example, the catheter 102 is 8 French. In another example, the catheter 102 is 10 French. Other sizes for the catheter 102 are possible as well.

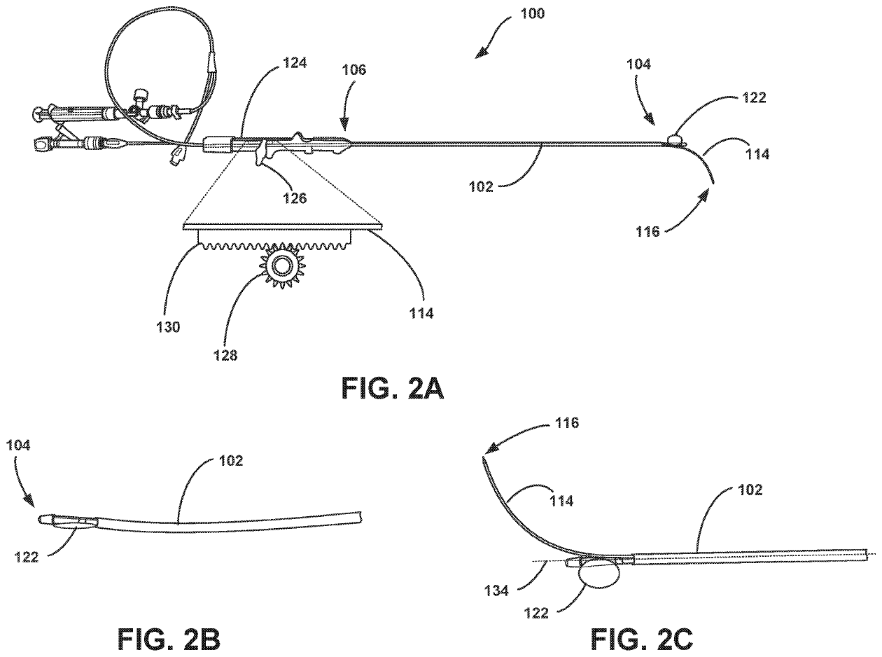

The device 100 also includes a needle 114 movably positioned within the second lumen 110 of the catheter 102. The needle includes a first end 116 and a second end 118 opposite the first end 116. The needle 114 is hollow such that the needle includes a lumen 120 extending from the first end 116 to the second end 118. The needle 114 is configured to transition from a first position in which the first end 116 of the needle 114 is positioned entirely within the second lumen 110 of the catheter 102 (shown in FIG. 2B) to a second position in which the first end 116 of the needle 114 extends out of the second lumen 110 of the catheter 102 (shown in FIG. 2C). The needle 114 has a non-zero radius of curvature in the second position.

The device 100 also includes a balloon 122 fixed to the first end 104 of the catheter 102. The balloon 122 is in fluid communication with the third lumen 112. In use, the third lumen is used for injecting and withdrawing solution of saline and x-ray contrast medium to inflate and deflate the balloon 122. The balloon 122 is eccentrically attached to the catheter 102 which functions to push the first end 104 of the catheter 102 against the wall of the hepatic vein and stabilize the needle 114 and catheter 102 when in use. The device 100 also includes a handle 124 coupled to the second end 106 of the catheter 102.

In one example, as shown in FIG. 2A, the handle 124 includes a knob 126. A rotation of the knob 126 in a first direction causes the needle 114 to transition from the first position to the second position, and a rotation of the knob 126 in a second direction opposite the first direction causes the needle 114 to transition from the seconds position to the first position. In one example, the knob 126 is coupled to a gear 128 positioned inside the handle 124 such that a rotation of the knob 126 corresponds to a rotation of the gear 128. In one such example, the gear 128 is in mechanical communication with a track 130 positioned inside the handle 124 such that a rotation of the gear 128 corresponds to a linear motion of the track 130. The track 130 is coupled to the needle 114 such that a linear motion of the track 130 corresponds to a linear movement of the needle 114 (e.g., from the first position to the second position and vice versa).

In one example, as shown in FIG. 2C, the first end 116 of the needle 114 extends away from a longitudinal axis 132 of the catheter 102 when the needle 114 is in the second position. In another example, as further shown in FIG. 2C, the balloon 122 is positioned on a first side of the catheter 102 and the first end 116 of the needle 114 extends away from a second side of the catheter 102 when the needle 114 is in the second position. The first side and the second side of the catheter 102 are positioned approximately 180 degrees from one another.

In one example, the second lumen 110 of the catheter 102 includes a rigid hollow member 134 positioned at the first end 104 of the catheter 102. In one particular example, the rigid hollow member 134 comprises a 19 gauge stainless steel cannula. The rigid hollow member 134 may help to stabilize the needle 114 and protect the catheter 102 from being damaged by the sharp tip of the needle 114 while the needle 114 is moved in and out of the second lumen 110.

In one example, the needle 114 is a 22 gauge needle. The needle 114 may comprise a shape-memory material that biases the needle 114 to a curved configuration in which the first end 116 of the needle 114 has a non-zero radius of curvature. As such, the needle 114 is in a straight configuration in the first position due to the constricting nature of the second lumen 110, and the needle 114 is in a curved configured in the second position due to the shape-memory properties of the first end 116 of the needle 114. In one example, the needle comprises a heat treated stainless steel material having a carbon content between about 0.4% and about 1.0%. Such a 22 gauge needle has resilience, shape-retaining properties to provide the non-zero radius of curvature in the second position, while also exhibiting sufficient hardness and strength to penetrate hard tissue such as a human cirrhotic liver. Alternatively, the needles may be constructed with a shape memory metal (e.g., commercially available memory metals, Nitinol, etc.), spring steel alloys, and similar metals.

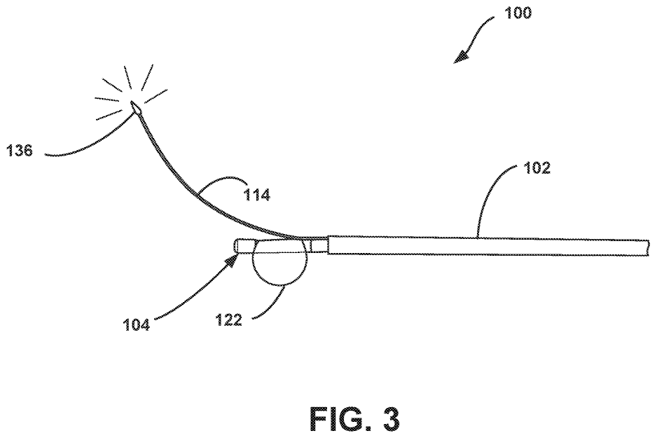

In another example, as shown in FIG. 3, the device 100 includes a light-emitting device 136 configured to be positioned through the lumen 120 of the needle 114 and extend out of the first end 116 of the needle 114. In one example, the light-emitting device emits a green light. In one specific example, the light-emitting device 136 comprises a 0.4 or 0.5 mm fiber optical borescope (InterTest, Inc., Columbia, NJ) that inserted into the lumen 120 of the needle 114, which will help to confirm the needle 114 in the portal vein. The use of fiber optical imaging using a green light to guide the puncture is based on the principle that hemoglobin completely absorbs green light. On the contrary, the liver reflects all the green light which can be acquired by the camera. Namely, when the first end 116 of the needle 114 is still in the liver, the light signal is very bright. Once the first end 116 of the needle 114 enters the portal vein, the signal immediately becomes completely dark, which instantly indicates the entry of the needle tip in the portal vein. The use of X-ray contrast medium (positive medium, such as Omnipaque or negative medium, such as CO2) will be minimized for the confirmation of the needle tip in the portal vein. The real-time optical imaging-guided technique will make the search for the portal vein simplified, much convenient and faster.

In one example, the 0.4 or 0.5 mm-diameter semi-rigid or flexible fiber optic borescope is sheathed by stainless steel or polyimide and has 3,000 picture elements. The fiber optic borescope may be inserted into the needle 114 through a “Y” gateway adaptor, which is connected to the second end 118 of the needle 114 and warrants the synchronic movement of the fiber optic scope with the needle 114. The light transmission fiber is connected to a green light source for transmitting the light to the distal end of the fiber optic scope to illuminate the target. The imaging fiber is connected to a scientific complementary metal-oxide-semiconductor (sCMOS) camera for detecting green light signal acquired by the imaging fiber.

In another example, the device 100 further includes an ultrasound catheter 138 configured to be removably positioned within the first lumen 108 of the catheter 102. The ultrasound catheter 138 can be moved and rotated synchronically within the first lumen 108 of the catheter 102 to find the portal vein. Since the field of view of the ultrasound image is oriented to the longitudinal profile of the first end 116 of the needle 114, the trajectory of the needle 114 can be visibly tracked, and in the meanwhile the in-plane ultrasound catheter 138 will locate the portal vein in the same image plane and facilitate the fast and precise puncture into the target. Such an arrangement may enable the surgeon to evaluate the positioning of the first end 116 of the needle 114 in real time to ensure proper placement of the needle 114 through the liver and into the portal vein.

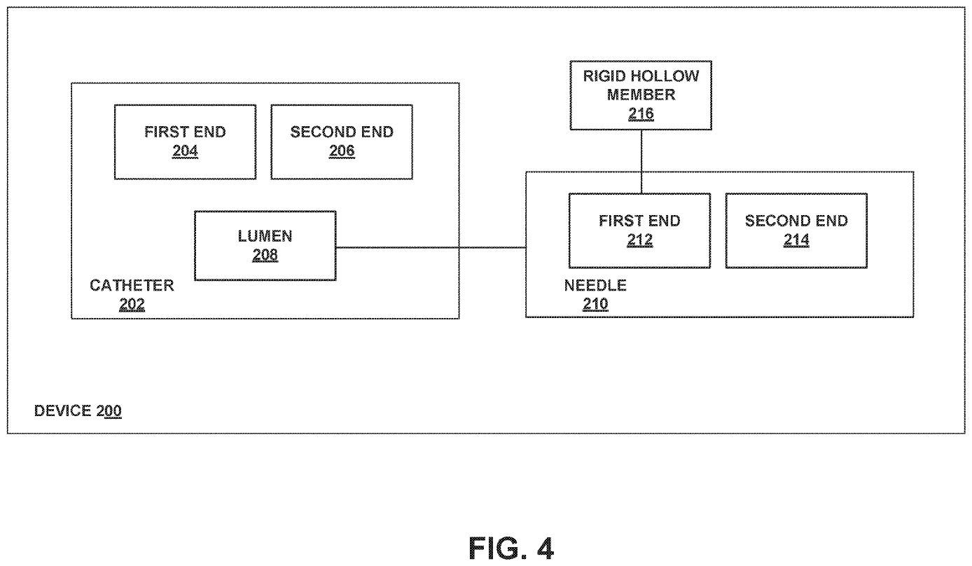

FIG. 4 illustrates a simplified block diagram containing an overview of the components of another device 200, according to an example embodiment. The device 200 includes a catheter 202 having a first end 204 and a second end 206 opposite the first end 204. The catheter 202 also includes a lumen 208. The device 200 also includes a needle 210 having a first end 212 and a second end 214 opposite the first end 212. The needle 210 is configured to be removably positioned within the lumen 208 of the catheter 202. The needle 210 is configured to transition from a first position in which the first end 212 of the needle 210 extends out of the lumen 208 of the catheter 202 to a second position in which the first end 212 of the needle 210 is positioned in the lumen 208 of the catheter 202. The device 200 further includes a rigid hollow member 216 fixed to and surrounding the needle 210 adjacent the first end. In one example, the needle 210 comprises a 22 gauge stainless steel needle with a tapered tip. In another example, the rigid hollow member 216 comprises a 19 gauge stainless steel tube. In another example, the catheter 202 is 5 French.

In use, the catheter 202 designed to complete a subsequent step in the TIPS procedure. The first end 204 of the catheter 202 and needle 210 combination is guided into the portal vein over a 0.018-inch guidewire which is placed in the portal vein via the device 100. The rigid hollow member 216 strengthens and supports the needle 210, fills the gap between the needle 210 and the wall of the catheter 202, and ensures the smooth transition between the needle 210 and catheter 202 as the needle 210 transitions from the first position to the second position.

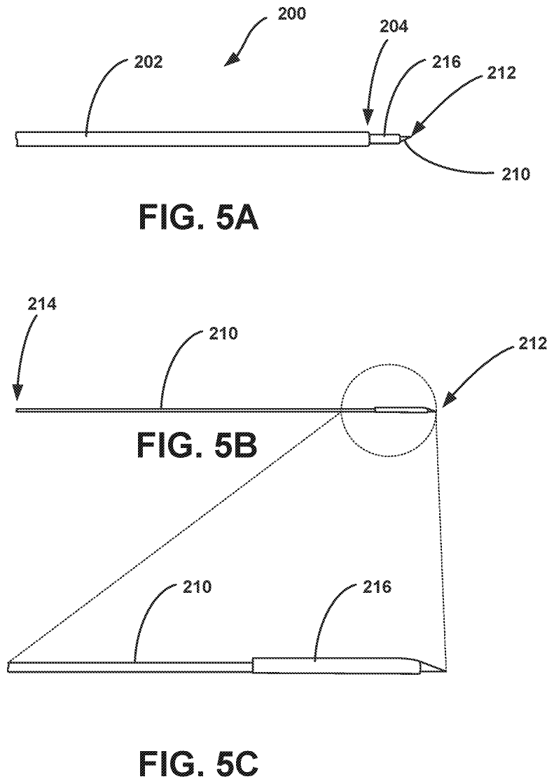

In one example, the present disclosure provides a system that includes the device 100 described above in relation to FIGS. 1-3 and the device 200 described above in relation to FIGS. 4-5C.

FIG. 6 is a block diagram of an example method for gaining access to a portal vein from a hepatic vein. Method 300 shown in FIG. 6 presents an embodiment of a method that could be used by the devices described above in relation to FIGS. 1-5C, as examples. Method 300 may include one or more operations, functions, or actions as illustrated by one or more of blocks 302-310. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 302, the method 300 includes positioning a first guidewire in the hepatic vein of a patient. The first guidewire may be positioned via a catheter, such as a 5 French catheter as a non-limiting example. In one example, the first guidewire comprises a 0.035-inch guidewire. At block 304, the method 300 further includes moving the device 100 described above in relation to FIGS. 1-3 over the first guidewire to the hepatic vein. At block 306, inflating the balloon 122 to fix the device 100 with respect to the hepatic vein. At block 308, the method 300 includes transitioning the needle 114 from the first position to the second position such that the first end 116 of the needle 114 moves through a liver of the patient and enters the portal vein. At block 310, the method 300 includes positioning a second guidewire through the lumen 120 of the needle 114 and into the portal vein. In one example, the second guidewire is a 0.018-inch guidewire.

In one example, the method 300 further includes (a) removing the device 100 described above in relation to FIGS. 1-3 and the first guidewire from the patient while keeping the second guidewire positioned in the portal vein, (b) moving the device 200 described above in relation to FIGS. 4-5C over the second guidewire to the portal vein, (c) removing the needle of the device 200 from the portal vein while keeping the catheter 202 in the portal vein, (d) positioning a third guidewire through the catheter 202 and into the portal vein, (e) moving a delivery catheter over the third guidewire and into the portal vein, and delivering a stent from the delivery catheter to thereby fluidically connect the portal vein to the hepatic vein.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A device comprising:
a catheter having a first end and a second end opposite the first end, wherein the catheter includes a first lumen, a second lumen, and a third lumen, and wherein the first lumen is configured to receive a guidewire therethrough to position the device in a desired anatomy.
a needle movably positioned within the second lumen of the catheter, wherein the needle includes a first end and a second end opposite the first end, wherein the needle is hollow such that the needle includes a lumen extending from the first end to the second end, wherein the needle is configured to transition from a first position in which the first end of the needle is positioned entirely within the second lumen of the catheter to a second position in which the first end of the needle extends out of the second lumen of the catheter, and wherein the needle has a non-zero radius of curvature in the second position;
a balloon fixed to the first end of the catheter, wherein the balloon is in fluid communication with the third lumen; and
a handle coupled to the second end of the catheter.

2. The device of claim 1, wherein the handle includes a knob, and wherein a rotation of the knob in a first direction causes the needle to transition from the first position to the second position, and wherein a rotation of the knob in a second direction opposite the first direction causes the needle to transition from the seconds position to the first position.

3. The device of claim 2, wherein the knob is coupled to a gear positioned inside the handle such that a rotation of the knob corresponds to a rotation of the gear, wherein the gear is in mechanical communication with a track positioned inside the handle such that a rotation of the gear corresponds to a linear motion of the track, and wherein the track is coupled to the needle.

4. The device of claim 1, wherein the first end of the needle extends away from a longitudinal axis of the catheter in the second position.

5. The device of claim 1, wherein the needle is a 22 gauge needle.

6. The device of claim 1, wherein the catheter is 8 French or 10 French.

7. (canceled)

8. The device of claim 1, wherein the second lumen of the catheter includes a rigid hollow member positioned at the first end of the catheter.

9. The device of claim 1, wherein the needle comprises a shape-memory material.

10. The device of claim 1, wherein the needle comprises a steel material having a carbon content between about 0.4% and 1.0%.

11. The device of claim 1, wherein the balloon is positioned on a first side of the catheter, wherein the first end of the needle extends away from a second side of the catheter in the second position, and wherein the first side is positioned approximately 180 degrees from the second side.

12. The device of claim 1, further comprising a light-emitting device configured to be positioned through the lumen of the needle and extend out of the first end of the needle.

13. The device of claim 12, wherein the light-emitting device emits a green light.

14. The device of claim 1, further comprising an ultrasound catheter configured to be removably positioned within the first lumen.

15. A device comprising:
a catheter having a first end and a second end opposite the first end, wherein the catheter includes a lumen;
a needle having a first end and a second end opposite the first end, wherein the needle is configured to be removably positioned within the lumen of the catheter, and wherein the needle is configured to transition from a first position in which the first end of the needle extends out of the lumen of the catheter to a second position in which the first end of the needle is positioned in the lumen of the catheter; and
a rigid hollow member fixed to and surrounding the needle adjacent the first end.

16. The device of claim 15, wherein the needle comprises a 22 gauge stainless steel needle.

17. The device of claim 15, wherein the rigid hollow member comprises a 19 gauge stainless steel tube.

18. The device of claim 15, wherein the catheter is 5 French.

19. A system comprising:
the device of claim 1; and
the device of claim 15.

20. A method for gaining access to a portal vein from a hepatic vein, the method comprising:
positioning a first guidewire in the hepatic vein of a patient;
moving the device of claim 1 over the first guidewire to the hepatic vein;
inflating the balloon to fix the device with respect to the hepatic vein;
transitioning the needle from the first position to the second position such that the first end of the needle moves through a liver of the patient and enters the portal vein; and
positioning a second guidewire through the lumen of the needle and into the portal vein.

21. The method of claim 20, further comprising:
removing the device of claim 1 and the first guidewire from the patient while keeping the second guidewire positioned in the portal vein;
moving the device of claim 15 over the second guidewire to the portal vein;
removing the needle of the device of claim 15 from the portal vein while keeping the catheter in the portal vein;
positioning a third guidewire through the catheter and into the portal vein;
moving a delivery catheter over the third guidewire and into the portal vein; and
delivering a stent from the delivery catheter to thereby fluidically connect the portal vein to the hepatic vein.
